(19) United States
(12) Patent Application Publication
(10) Pub. No.: US 2021/0145930 A1
(43) Pub. Date: May 20, 2021

(54) MULTIPLE LAYER ARTICLE WITH INTERACTIVE REINFORCEMENTS LINEAR RIBBON FIBER REINFORCEMENT FOR COMPOSITE FORMS

(71) Applicant: NeuroTheranostics, Inc., Detroit, MI (US)

(72) Inventors: John Anagli, Detroit, MI (US); Tracy Anagli, Detroit, MI (US)

(73) Assignee: NeuroTheranostics, Inc., Detroit, MI (US)

(21) Appl. No.: 17/046,839

(22) PCT Filed: May 7, 2019
(86) PCT No.: PCT/US2019/031103

§ 371 Date: Oct. 12, 2020

Related U.S. Application Data
(60) Provisional application No. 62/667,886, filed on May 7, 2018.

(51) Int. Cl. A61K 38/17; A61K 47/26; A61K 47/12

(52) U.S. Cl. CPC A61K 38/17; A61K 47/12; A61K 47/26

(57) ABSTRACT

A method and composition are provided to treat traumatic brain injury and inhibit the cascade of secondary injury neuronal damage in a patient following traumatic brain injury. The method and composition are especially advantageous for immediate treatment at the situs of the traumatic brain injury. The method includes treating the traumatic brain injury by administering at least one effective dose of a peptidomimetic calpain inhibitor to the upper one-third of the patients nasal cavity, thereby enabling the at least one effective dose of peptidomimetic calpain inhibitor to bypass the patients blood-brain barrier and delivering the at least one effective dose of peptidomimetic calpain inhibitor to the patients central nervous system. The composition includes at least one effective dose of a peptidomimetic calpain inhibitor having a synthetic peptide of SEQ ID NOS: 2, 3, 5, 6, 7, 8, 9, or 10.

### CROSS-REFERENCE TO RELATED APPLICATION

This application claims priority of United States Provisional Patent Application Ser. No. 62/667,886 filed May 7, 2018, which is incorporated herein by reference.

### TECHNICAL FIELD

The present invention generally relates to compositions and methods for treating traumatic brain injury, and more specifically to a protein peptide or protein neurotherapeutic for delivery to a victim of a traumatic brain injury in the form of a nasal spray or other pharmaceutically acceptable vehicle or carrier to mitigate a cascade of secondary injury.

### BACKGROUND

Traumatic brain injury (TBI) occurs in mammals, including in humans, when a sudden trauma causes damage to the brain. TBI can result when the head suddenly and violently hits an object, blast-induced neurotrauma can result from the blast waves without impact with a physical object or when an object pierces the skull and enters brain tissue. Symptoms of TBI include a loss of consciousness, headache, confusion, lightheadedness, dizziness, blurred vision or tired eyes, ringing in the ears, bad taste in the mouth, fatigue or lethargy, a change in sleep patterns, behavioral or mood changes, trouble with memory, concentration, attention, or thinking, vomiting or nausea, convulsions or seizures, an inability to awaken from sleep, dilation of one or both pupils of the eyes, slurred speech, weakness or numbness in the extremities, loss of coordination, and increased confusion, restlessness, and agitation. Symptoms of a TBI can be mild, moderate, or severe, depending on the extent of the damage to the brain.

Secondary injuries resulting from a TBI depend upon the severity of the injury, the location of the injury, the age and general health of the individual, and how quickly the individual receives treatment. Some common secondary injuries that result from TBI include problems with cognition (thinking, memory, and reasoning), sensory processing (sight, hearing, touch, taste, and smell), communication (expression and understanding), and behavior or mental health (depression, anxiety, personality changes, aggression, acting out, and social inappropriateness). More serious head injuries may result in stupor, an unresponsive state, but one in which an individual can be aroused briefly by a strong stimulus, such as sharp pain; coma, a state in which an individual is totally unconscious, unresponsive, unaware, and unarousable; vegetative state, in which an individual is unconscious and unaware of his or her surroundings, but continues to have a sleep-wake cycle and periods of alertness; and a persistent vegetative state (PVS), in which an individual stays in a vegetative state for more than a month.

Anyone with signs of moderate or severe TBI should receive medical attention as soon as possible to reduce the cascade of secondary injury. Because little can be done to reverse the initial brain damage caused by trauma, medical personnel try to stabilize an individual with TBI and provide treatment to prevent the cascade of secondary injury. Primary concerns include insuring proper oxygen supply to the brain and the rest of the body, maintaining adequate blood flow, and controlling blood pressure.

Treatment of TBI typically includes providing a neurotherapeutic to the injured individual. Such therapeutics are typically administered one of two ways: intravenously or through a catheter placed directly into the brain through a drilled hole in the skull, both of which present a number of undesirable problems. First, while intravenous administration of TBI treatment therapeutics is minimally invasive and atraumatic, it is undesirably ineffective in that much of the therapeutic is passed through the body without treating the TBI because the therapeutic is unable to cross the blood-brain barrier that protects the brain from the unregulated leakage and entry of substances, including proteins, from the blood. Similarly, direct introduction of a therapeutic into the brain through a catheter is often undesirable because of the additional trauma caused by drilling through the skull to place the catheter. Additionally, because both intravenous and direct catheter delivery of a treatment therapeutic require administration by a medical professional and sterile medical equipment, a significant amount of time elapsed before the injured individual receives treatment, thereby increasing the chance and severity of secondary injuries.

Accordingly, there exists a need in the art for a way to immediately administer a neurotherapeutic at the situs of injury (e.g. sports field, battle field, or vehicle accident) to treat TBI and mitigate the cascade of secondary injury.

### SUMMARY OF THE INVENTION

A method of treating traumatic brain injury is provided for inhibiting the cascade of secondary injury neuronal damage in a patient following traumatic brain injury. The method includes treating the traumatic brain injury by administering at least one effective dose of a peptidomimetic calpain inhibitor to the upper one-third of the patient's nasal cavity, thereby enabling the at least one effective dose of peptidomimetic calpain inhibitor to bypass the patient's blood-brain barrier and delivering the at least one effective dose of peptidomimetic calpain inhibitor to the patient's central nervous system. The at least one effective dose of a peptidomimetic calpain inhibitor can be administered alone or in combination with a pharmaceutically acceptable excipient, carrier, or other vehicle.

A neurotherapeutic composition is also provided for inhibiting the cascade of secondary injury neuronal damage following traumatic brain injury. The composition includes at least one effective dose of a peptidomimetic calpain inhibitor having a synthetic peptide of SEQ ID NOS: 2, 3, 5, 6, 7, 8, 9, or 10. The composition may also include a pharmaceutically acceptable excipient or other additive including at least one of a liquid spray, a powdered spray, nose drops, a gel, an ointment, and combinations thereof. The pharmaceutically acceptable excipient or other additive may be selected from the group consisting of a hydrophobic blood-brain-barrier or a membrane-permeant amino acid sequence linked by a polyethylene glycol linker to the N-terminus of said peptide, SEQ ID NO. 11, SEQ ID NO. 12, a nonaqueous vehicle, nanoparticle encapsulation, a solvent, a diluent, adjuvant, a chelating agent, a stability enhancing additive, antimicrobial preservatives, antioxidants, buffers, an antibacterial or an antifungal agent, parabens, chlorobutanol, phenol, sorbic acid, an isotonic agent, a sugar, sodium chloride, a prolonged absorption agent, aluminum monostearate, a gelatin, and combinations thereof.

### BRIEF DESCRIPTION OF THE DRAWINGS

FIGS. 1A-K are micrographs and density plots that show delayed (24 h post injury) SEQ ID NO. 5 treatment significantly increased cell proliferation (BrdU⁺ cells), neurogenesis (newborn mature neurons) and vascular density in the ipsilateral cortex at 25 days after mild-moderate TBI in rats: in which:

FIGS. 1A and B show red fluorescent labeling of BrdU⁺ cells showing treatment significantly increased the number of BrdU-positive cells in the injured cortex (FIG. 1B) compared to the saline-treated group (FIG. 1A), the number of BrdU⁺ cells is shown in FIG. 1C.

FIGS. 1E and 1H show blue fluorescence labeling of cell nuclei with Dapi. Co-localization of the BrdU-positive nuclei within MAP2-labeled was found (FIG. 1H) indicating the presence of newborn mature neurons. SEQ ID NO. 5-treatment significantly increased the number of newborn neurons in the ipsilateral DG 25 days post injury (FIGS. 1D-H) compared to the saline controls. The bar graph in FIG. 1F shows the number of newborn neurons counted in the DG and expressed per mm².

FIGS. 1I and 1J show EBA-stained vascular structure, rats treated with SEQ ID NO. 5 had significantly increased density (vessels/mm2) of EBA positive vessels in the injured cortex area (p<0.05) (FIG. 1J), compared with saline-treated rats (FIG. 10. The density of EBA-stained vasculature in the injured cortex is shown in FIG. 1K.

FIGS. 2A-C are plots that demonstrate delayed (24 h post injury) SEQ ID NO. 5 treatment significantly improved neurological functional recovery;

FIG. 2A shows that significantly improved sensorimotor scores measured at Days 14-25 after TBI in the SEQ ID NO. 5-treated group compared to the saline-treated group (P<0.05);

FIG. 2B shows the percentage of time spent in the correct quadrant (Northeast) by all TBI rats gradually increased from Days 21 to 25 after surgery. As compared to the control (saline-treated) group, the SEQ ID NO. 5 treated group showed a significant improvement in spatial learning and memory retention at Days 23 to 25 post-injury (3rd to 5th day of MWM testing) (P<0.05). As compared to preinjury baseline, the incidence of contralateral right forelimb footfaults significantly increased after TBI; and

FIG. 2C is a plot that shows treatment with SEQ ID NO. 5 showed no significant improvement in neurological function over the control group, assessed by the footfault test.

### DETAILED DESCRIPTION OF THE INVENTION

The present invention has utility as compositions and methods for treating traumatic brain injury, and more specifically as a protein neurotherapeutic for delivery to a victim of a traumatic brain injury in the form of a nasal spray to mitigate a cascade of secondary injury. The nasal spray may be delivered at the situs of injury for rapid delivery of the therapeutic to the victim. The protein is robust and amenable to storage. The nasal spray also has advantages in bringing the blood-barrier transiting protein into immediate proximity to brain tissue.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

As used herein, “central nervous system” (CNS) refers to the brain and spinal cord and associated tissues.

An “effective amount” of agent is an amount sufficient to prevent, treat, reduce and/or ameliorate the symptoms, neuronal damage and/or underlying causes of any of traumatic brain injury.

In the context of the present invention, the terms “treat” and “therapy” and “therapeutic” and the like refer to alleviate, slow the progression, prophylaxis, or attenuation of the cascade of secondary injury resulting from traumatic brain injury.

“Prevent,” as used herein, refers to putting off, delaying, slowing, inhibiting, or otherwise stopping, reducing or ameliorating the secondary injury associated with traumatic brain injury. It is preferred that a large enough quantity of the agent be applied in non-toxic levels in order to provide an effective level of neuroprotection. The method of the present invention may be used with any animal, such as a mammal or a bird (avian), more preferably a mammal. Poultry are a preferred bird. Exemplary mammals include, but are not limited to rats, mice, cats, dogs, horses, cows, sheep, pigs, and more preferably humans.

Thus, methods and pharmaceutical compositions are described herein that, inter alia, prevent, and/or treat neurologic complications such as cognitive, behavioral and/or physical impairment due to traumatic brain injury.

The present invention provides embodiments containing peptidomimetic versions of compounds that contain a number of non-peptidic chemical bonds, non-proteinogenic amino acids, and/or conformational constraints.

Embodiments of the present invention provide a nucleotide as well as related amino acid sequence of the native 27-amino acid calpastatin-derived peptide to develop peptidomimetic calpain inhibitors of some embodiments, as follows:

(SEQ ID NO: 1)
ct cca aaa tat agg gaa eta ttg get
(SEQ ID NO: 2)
Asp Pro Met Ser Ser Thr Tyr Ile Glu Glu Leu Gly
Lys Arg Glu Val Thr Ile Pro Pro Lys Tyr Arg Glu
Leu Leu Ala

In some embodiments, side-chain to side-chain cyclization is performed between Lys21 and Glu24 side chains of SEQ ID No. 2.

Some embodiments comprise the following natural amino acid replacements in SEQ ID NO: 2:

Arg²³ to Va²³

Glu²⁴ to Ala²⁴

Ala²⁷ to Pro²⁷,

thus providing a sequence Asp Pro Met Ser Ser Thr Tyr Ile Glu Glu Leu Gly Lys Arg Glu Val Thr Ile Pro Pro Lys Tyr Val Ala Leu Leu Pro (SEQ ID NO: 3). In some embodiments, the hydrophobic sequence Ala Val Leu Leu Ala Leu Leu Ala Pro (SEQ ID NO: 4) is added to the C-terminus of SEQ ID NO: 3 to provide a sequence of Asp Pro Met Ser Ser Thr Tyr Ile Glu Glu Leu Gly Lys Arg Glu Val Thr Ile Pro Pro Lys Tyr Val Ala Leu Leu Pro Ala Val Leu Leu Ala Leu Leu Ala Pro (SEQ ID No: 5).

In some embodiments, one or more of the more usual peptidic bonds between the following amino acid pairs of SEQ ID NO: 2, SEQ ID NO: 3, or SEQ ID NO. 5 are replaced with non-peptidic reduced amide bonds rendering the peptidomimetic resistant to cleavage by proteolytic enzymes in vivo, and consequently improving its pharmacokinetic properties:

Ser4-Ser5

Tyr6-Ile7

Lys13-Arg¹⁴

Glu¹⁵-Val¹⁶

Lys²¹-Tyr²²

Tyr²²-Arg²³

Arg²³-Glu²⁴.

Moreover, in some embodiments, one or more of the following natural to un-natural amino acid substitutions are made in SEQ ID NO: 2, SEQ ID No: 3, or SEQ ID NO: 5:

Ile18-+Nval18

Arg²³-+Orn²³ providing sequences:

(SEQ ID NO: 6)
Asp Pro Met Ser Ser Thr Tyr Ile Glu Glu Leu Gly
Lys Arg Glu Val Thr Nva Pro Pro Lys Tyr Orn Glu
Leu Leu Ala;
(SEQ ID NO: 7)
Asp Pro Met Ser Ser Thr Tyr Ile Glu Glu Leu Gly
Lys Arg Glu Val Thr Nva Pro Pro Lys Tyr Arg Glu
Leu Leu Ala;
(SEQ ID NO: 8)
Asp Pro Met Ser Ser Thr Tyr Ile Glu Glu Leu Gly
Lys Arg Glu Val Thr Ile Pro Pro Lys Tyr Orn Glu
Leu Leu Ala;
(SEQ ID NO: 9)
Asp Pro Met Ser Ser Thr Tyr Ile Glu Glu Leu Gly
Lys Arg Glu Val Thr Nva Pro Pro Lys Tyr Val Ala
Leu Leu Pro;
or
(SEQ ID No: 10)
Asp Pro Met Ser Ser Thr Tyr Ile Glu Glu Leu Gly
Lys Arg Glu Val Thr Nva Pro Pro Lys Tyr Val Ala
Leu Leu Pro Ala Val Leu Leu Ala Leu Leu Ala Pro.

In some embodiments, without limitation, the compounds contain a hydrophobic blood-brain-barrier (“BBB”) or membrane-permeant amino acid sequence linked by a polyethylene glycol linker to the N-terminus of the peptidomimetic inhibitor. Without limitation, one example of a hydrophobic membrane-permeant amino acid sequence, and related nucleotide sequence, comprising embodiments of the invention as follows:

(SEQ ID NO: 11)
gee gcg gta gcg ctg etc ccg gcg gtc ctg ctg gee
ttg ctg gcg ccc;
(SEQ ID NO: 12)
Ala Ala Val Ala Leu Leu Pro Ala Val Leu Leu Ala
Leu Leu Ala Pro.

Non-peptidic BBB permeant structures such as taurine, cholesterol, a nonionic amphiphilic diethylene glycol methyl moiety, etc. can also be optionally attached to the inhibitor.

Dysregulation of calpain, a cysteine proteinase recognized to play an important role in signal transduction, cell migration and regulation of apoptosis, has been implicated in a variety of neurodegenerative disorders, including tissue damage, following stroke, traumatic brain and spine injury. These disorders are characterized by intracellular calcium overload leading to excessive activation of calpains. In vivo inhibition of calpain using cysteine protease inhibitors (e.g. peptide aldehydes, alpha-keto esters and amides) diminishes the extent of neuronal damage following ischemia or traumatic brain injury; however, the relative nonspecificity of these cysteine proteinase inhibitors precludes a definitive role for calpain participation. Calpastatin is the only inhibitor that is specific for calpain, and it is generally accepted that the interaction of calpastatin with calpain is the most relevant mechanism responsible for the regulation of Ca2 +-induced proteolysis. The deduced primary structure of calpastatin consists of a non-inhibitory L-domain and four repeating inhibitory domains, each having an independent inhibitory activity against calpain and thus constituting a functional unit (See Emori et al, “All four repeating domains of the endogenous inhibitor for calcium-dependent protease independently retain inhibitory activity. Expression of the cDNA fragments in Escherichia coli”, J Biol. Chem. 263, 2364-2370).

Investigation of the pathobiology of cerebral ischemia or trauma indicates that the initial ischemic or traumatic insult induces massive release of glutamate from damaged synapses which leads to activation of glutamate receptor-associated and voltage-dependent calcium channels. Such activation induces influx of calcium ions into the neuron and release of calcium ions from intracellular stores. Loss of intracellular calcium homeostasis contributes to cell death by activating various enzymes, including proteases, kinases, phosphatases, and phospholipases. Integral to the mechanism of calcium-mediated brain injury is the pathologic activation of calpains. Under normal physiological conditions, calpain exists at very low activity in cells and is proposed to participate in the turnover of cytoskeletal proteins and the regulation of kinases, transcription factors, and receptors. However, pathologic calpain activation results in proteolytic destruction of many cellular proteins including receptor proteins, calmodulin binding proteins, signal transduction enzymes, transcription factors, and cytoskeletal proteins. Furthermore, uncontrolled calpain activity prevents increased expression of several key proteins, including growth-associated protein-43 (GAP-43), synaptophysin, and collapsin receptor mediator proteins (CRMPs), that play a major role in regeneration and neuroplasticity after ischemic and traumatic brain injury. Thus, calpains can contribute to the pathogenesis of ischemic or traumatic brain injury via multiple molecular and cellular pathways. Thus, selective inhibition of calpains may lead to both cerebroprotective effects and an enhancement of neuronal plasticity/repair mechanisms.

There are two “hot spots” within the calpastatin molecule in which side chains of the most critical residues of calpastatin interact with hydrophobic pockets in calpain. Mutation of any of the key residues in either hot spot results in loss of inhibitory activity. Different regions in the inhibitory domain of calpastatin interact with complementary sites in calpain and undergo calpain-induced conformational changes that are crucial to inhibitor's affinity and selectivity. Results indicate that specific inhibition of calpain in vivo by administration of novel membrane- permeant calpastatin peptide(s) reduces infarct volume and neurological deficits by blocking post-ischemic or traumatic proteolysis of vital brain proteins.

Findings substantiate the involvement of calpain in postischemic neurological and cerebrovascular dysfunction and show a protective mechanism of SEQ ID NO: 5. The protective mechanism is, in part, due to preservation of cerebral endothelial function. An interesting finding is that calpain is involved in regulation of endothelial nitric oxide synthase, a key enzyme involved in neurovascular function, through phosphorylation events. SEQ ID NO: 5 as a novel BBB-permeant specific inhibitor of calpain provides a novel approach for the treatment of traumatic brain injury.

Thus, without limitation and without disclaimer of subject matter, some embodiments comprise novel compositions and methods to prevent, control, or alleviate mammalian injury, including without limitation, brain damage, through the selective application of novel calpain inhibitors. In accordance with some embodiments, without limitation, one may inhibit such trauma through the administration of one or more such inhibitors for a finite interval of time, thereby limiting the development of such damage.

In accordance with some embodiments, there is a high likelihood that the duration of drug therapy would be relatively brief and with a high probability of success. Prophylactic administration of efficacious amounts of the calpain inhibitors of some embodiments may greatly reduce the incidence of damage associated with many forms of neural trauma.

The calpain inhibitor(s) of some embodiments would be administered and dosed in accordance with good medical practice, taking into account the clinical condition of the individual patient, the site and method of administration, scheduling of administration, patient age, sex, body weight and other factors known to medical practitioners. The “pharmaceutically effective amount” for purposes herein is thus determined by such considerations as are known in the art. The amount must be effective to achieve improvement, including but not limited to, decreased damage or injury, or improvement or elimination of symptoms and other indicators as are selected as appropriate measures by those skilled in the art.

In accordance with some embodiments, such calpain inhibitor(s) can be administered in various ways. It can be administered alone or as an active ingredient in combination with pharmaceutically acceptable carriers, diluents, adjuvants, and vehicles. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

According to various embodiments of the present disclosure, a patient that has undergone a traumatic brain injury is treated by administering at least one dose of a therapeutic agent via intranasal delivery to the upper one-third of the nasal cavity. The effects of post-TBI intranasal administration of such a therapeutic agent include significant reduction of infarct volume as well as a significant decrease in secondary injury associated with TBI.

Rather than invasive and traumatic intracranial administration and generally ineffective intravenous injection of a therapeutic agent, the present invention provides a non-invasive method to directly target the substance to the brain and thus the central nervous system (CNS). Intranasal delivery allows substances to be rapidly delivered to the central nervous system, even those that do not readily cross the blood-brain barrier by bypassing the blood-brain barrier and directly exposes the CNS to the delivered substance. In this manner, unwanted systemic side effects are reduced if not eliminated.

According to inventive embodiments of the present disclosure, a method delivers the therapeutic agent to the nasal cavity of a mammal. It is preferred that the agent be delivered to the olfactory area in the upper one-third of the nasal cavity and, particularly, to the olfactory neuroepithelium in order to promote rapid and efficient delivery of the agent to the CNS along the olfactory neural pathway rather than the capillaries within the respiratory epithelium. The preferred transport of the therapeutic agent to the brain by means of the olfactory and trigeminal neural pathways rather than the circulatory system so that the harmful side effects and potentially short half-life of the agent is not an issue. Further, certain agents may simply be unable due to size to cross the blood-brain barrier from the bloodstream into the CNS. The preferred method allows direct delivery of such molecules to the CNS. The inventive method provides increased efficacy of the therapeutic agent and rapid delivery of the therapeutic agent soon after the occurrence of a traumatic brain injury.

To deliver the therapeutic agent to the CNS, the agent alone or in combination with other substances as a pharmaceutical composition may be administered to the olfactory area located in the upper one-third of the nasal cavity. The composition may be administered intranasally as a powdered or liquid spray, nose drops, a gel or ointment, through a tube or catheter, by syringe, packtail, pledget or by submucosal infusion. Optimization of the administration of the therapeutic agent is provided by the various embodiments by applying the agent to the upper third of the nasal cavity.

The optimal concentration of the active therapeutic agent will necessarily depend upon the specific neurologic agent used, the characteristics of the patient and the nature of the disease or condition for which the agent is being used.

In general, any of the therapeutic agents or pharmaceutical compositions described or referenced herein may be administered to patients or subjects under embodiments of the inventive method to treat patients at risk for, or diagnosed with, traumatic brain injury, spinal cord injury or cerebral hemorrhage.

An effective amount, as herein defined, of the therapeutic agent to be administered pursuant to embodiments of the invention is the most preferred method of expression of dosage. Such effective amount is dependent upon many factors, including but not limited to, the type of disease or condition giving rise to an anticipated cerebral ischemic or traumatic episode, the patient's general health, size, age, and the nature of treatment, i.e., short-term or chronic treatment. For illustrative purposes only, exemplary treatment regimens relating generally to the therapeutic agents disclosed herein, including dosage ranges, volumes and frequency are provided below:

Efficacious dosage range: 0.0001-1.0 mg/kg (milligram per kilogram of body weight).

A more preferred dosage range may be 0.005-1.0 mg/kg.

The most preferred dosage range may be 0.05-1.0 mg/kg.

The dosage volume (applicable to nasal sprays or drops) range may be 0.015 ml-1.0 ml.

The preferred dosage volume (applicable to nasal sprays or drops) range may be 0.03 ml-0.6 ml.

Generally, the treatment may be given in a single dose or multiple administrations, i.e., once, twice, three or more times daily over a period of time.

The brain concentrations that are likely to be achieved with the dosage ranges provided above are, for a single dose: 0.1 nM-50 μM. Over the course of a multi-dose treatment plan, the maximum brain concentration may be as high as 500 μM.

### EXAMPLES

The present invention is further detailed with respect to the following examples. These examples are non-limiting and not intended to limit the scope of the appended claims.

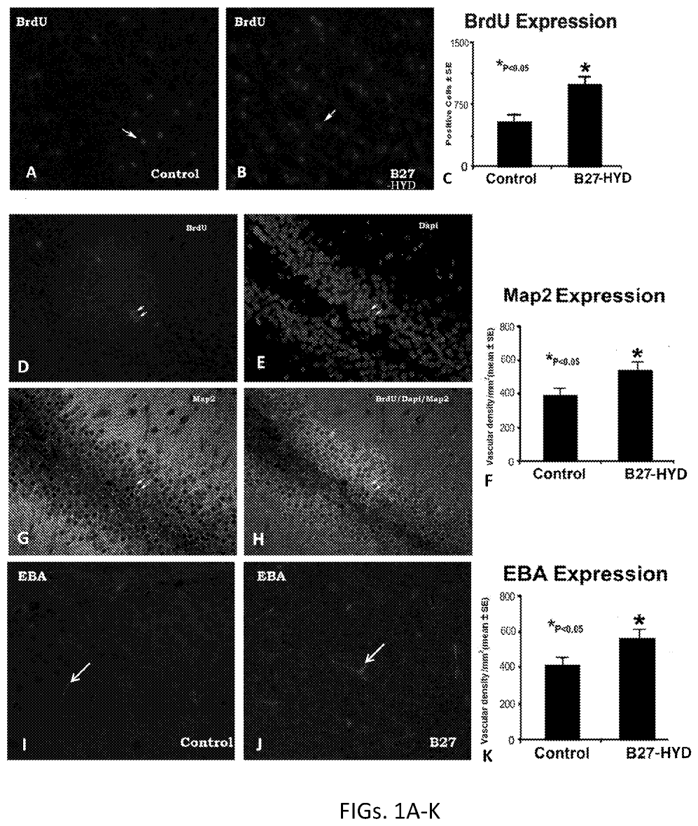

FIGS. 1A-K: SEQ ID NO. 5 treatment significantly increased cell proliferation in the ipsilateral cortex and neurogenesis (increase in newborn mature neurons) in the dentate gyrus at 25 days after mild-moderate TBI.

BrdU, an analog of thymidine, can be incorporated into the newly synthesized DNA of replicating cells during the S phase of the cell cycle, substituting for thymidine during DNA replication. BrdU immunostaining is commonly used to detect cell proliferation. To determine the effect of SEQ ID NO. 5 (3 mg/kg, i.v.) or saline on cell proliferation, BrdU (100mg/kg) was injected i.p. daily starting at day 1 after mild-moderate TBI and continued for 10 days after TBI to label newly generated cells. Twenty-five days after TBI, the animals were anesthetized and sacrificed. The brains were fixed in 4% paraformaldehyde and cut into seven equally spaced 2-mm coronal blocks using a rat brain matrix. The number of BrdU-positive cells found in the ipsilateral cortex and hippocampus was significantly increased in the SEQ ID NO. 5-treated rats examined at 25 days after TBI, compared to the saline controls. FIGS. 1A-B show red fluorescent labeling of BrdU⁺ cells (arrow as example of BrdU⁺ cells). The number of BrdU-positive cells found in the ipsilateral cortex and hippocampus was significantly increased in the SEQ ID NO. 5 treated rats examined at 25 days after TBI, compared to the saline controls. SEQ ID NO. 5 treatment significantly increased the number of BrdU-positive cells in the injured cortex (FIG. 1B) compared to the saline-treated group (FIG. 1A). The number of BrdU⁺ cells is shown in FIG. 1C. Data represent mean±SE. *p<0.05 vs saline group. n (rats/group)=7.

A large part of the functional recovery that occurs after brain injury is due to neurogenesis, and brain plasticity caused by anatomical and functional reorganization of the CNS. To determine the effect of SEQ ID NO. 5 on neurogenesis, the brain blocks containing the hippocampus were processed for vibratome sections (100 μm) followed by double fluorescence staining for endogenous cellular proliferation {BrdU positive cells (red) and MAP 2 (green) to identify newborn neurons in the ipsilateral dentate gyrus (DG) of the hippocampus. Five sections with 100-μm intervals from the dorsal dentate gyms were used for immunohistochemical staining with the same antibody. Sections were blocked in a Tris-buffered saline solution containing 5% normal goat serum, 1% BSA and 0.05% Tween-20. Sections were then incubated with the primary antibodies for localization of BrdU (1: 100; a marker for proliferation cells) and MAP-2 (microtubule-associated protein-2) (1:500, a marker for neuron) and EBA (endothelial barrier antigen) (1:1000, a marker for detection of mature vessels). The sections were incubated with Cy3- and/or FITC-conjugated antibody (1:200; Jackson ImmunoResearch) at room temperature for 2 hours. Quantitative measurements of immunostaining were performed by an observer blinded to the individual treatment status of the animals. All slides were digitized under a x20 objective lens (Nikon, Eclipse 80i, Melville, N.Y.) by using a CoolSNAP color camera (Photometrics, Tucson, Ariz.) interfaced with a MetaMorph image analysis system (Molecular Devices, Downingtown, Pa.). The cells with BrdU that clearly localized to the nucleus were counted as BrdU-positive cells. BrdU-positive cells in the lesion boundary zone and BrdU/MAP-2 positive cells in the dentate gyrus were digitized. The fluorescent images were analyzed with a Bio-Rad MRC 1024 (argon and krypton) laser-scanning confocal imaging system mounted onto a Zeiss microscope (Bio-Rad, Cambridge, Mass.). Blue fluorescence labeling of cell nuclei with Dapi is shown in FIGS. 1E and 1H. Co-localization of the BrdU-positive nuclei within MAP2-labeled was found (FIG. 1H) indicating the presence of newborn mature neurons. SEQ ID NO. 5-treatment significantly increased the number of newborn neurons in the ipsilateral DG 25 days post injury (FIGS. 1D-H) compared to the saline controls. The bar graph in FIG. IF shows the number of newborn neurons counted in the DG and expressed per mm². Data represent mean±SE. *p<0.05 vs saline group, n (rats/group)=7.

Endothelial barrier antigen (EBA)-staining has been used to identify vascular structure in the brain after ischemic brain injury. To evaluate the effect of SEQ ID NO. 5-therapy on vessel proliferation and vascular density, quantitative measurement of Endothelial Barrier Antigen (EBA) staining was performed. For quantification of vessels, the number of EBA positive vessels was measured in eight fields of view within the lesion boundary zone area. Data are presented as the density of EBA immunoreactive vessels relative to the area of the lesion boundary zone. Arrows in FIGS. 1I-J show EBA-stained vascular structure. When compared to controls, rats treated with SEQ ID NO. 5 had significantly increased density (vessels/mm2) of EBA positive vessels in the injured cortex area (p<0.05) compared with saline-treated rats. The density of EBA-stained vasculature in the injured cortex is shown in the right panel. Data represent mean±SE. *p<0.05 vs saline group. n (rats/group)=7.

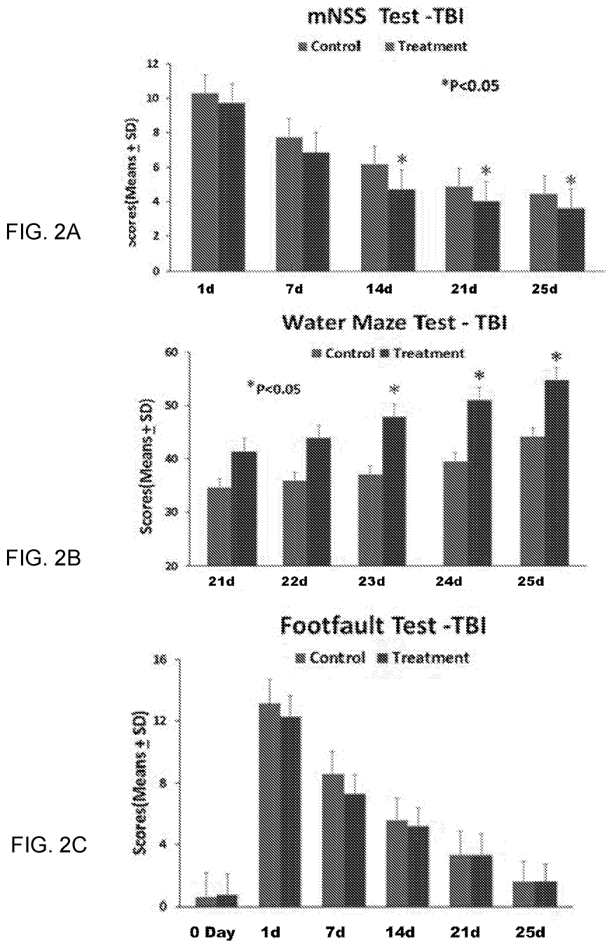

FIGS. 2A-C: Delayed (24 h post injury) SEQ ID NO. 5-treatment significantly improved neurological functional recovery after mild-moderate TBI.

A controlled cortical impact (CCI) device was used to induce mild-moderate TBI. Adult male (280-300 g) Sprague-Dawley rats (Harlan: Indianapolis, Ind.) were anesthetized intraperitoneally with xylazine (10 mg/kg body weight) and ketamine (40 mg/kg body weight). Rectal temperature was maintained at 37° C. using a feedback-regulated water-heating pad. The surgical area of the skull was prepared using germicidal povidone-iodine scrub followed by a 70% isopropyl alcohol rinse combinations, being careful to scrub from the center of the surgical site toward the periphery. Rats were placed in a stereotactic frame. Two 10-mm-diameter craniotomies were performed adjacent to the central suture, midway between lambda and bregma. The second craniotomy allowed for lateral movement of cortical tissue. The dura mater was kept intact over the cortex. Injury was delivered by impacting the left cortex (ipsilateral cortex) with a 5 mm diameter aluminum impactor tip (housed in a pneumatic cylinder) at a velocity of 3.5 m/s with a 1.0 mm (mild-moderate) compression and 150 ms dwell time (compression duration). Velocity was measured with a linear velocity displacement transducer. After the TBI, animals were randomly divided into control group (n=10) and treated group (n=10). SEQ ID NO. 5 (i.v. slow bolus injection; 3 mg/kg) or control solution was administered by tail vein at 24 hours and 48 hours. Animals were sacrificed at 25 days after treatment. Neurological functional outcome was measured by the modified neurological severity score (mNSS), Morris Water Maze (MWM) and foot-fault test. mNSS is a composite of the motor (muscle status, abnormal movement), sensory (visual, tactile and proprioceptive) and reflex tests. The mNSS is used to assess neurological functions by a 0-18 composite score of motor, sensory, balance, and reflex measures, with higher scores implying greater neurological injury. Morris Water maze (MWM) was used to monitor cognitive (spatial learning) improvement. MWM testing was performed during the 21-25 days after experimental TBI. Data collection was automated by the HVS Image 2020 Plus Tracking System (US HVS Image). Each rat was given four trials per day for 4 days with a hidden platform at a fixed location, and then 4 trials a day for 5 days with the platform in a different location hidden beneath the surface of the water. Each trial did last a maximum of 90 sec. If the animal reached the platform within 90 sec, the percentage of time traveled within the NE (correct) quadrant was calculated relative to the total amount of time spent swimming before reaching the platform and employed for statistical analysis. The platform was 11 cm in diameter; the pool (123 cm diameter ×32 cm height). The temperature of the water in the tank was close to 26° C. Rats were monitored continuously while in the tank. After testing the rats were towel-dried and recovered in a warm recovery box with a warming pad and drape. The foot-fault test was used to evaluate sensorimotor function. Rats were tested for placement dysfunctions of forelimbs with the modified foot-fault test. Briefly, animals were placed on elevated hexagonal grids of different sizes. Animals tend to move on the grid with their paws placed on the wire frame. When animals inaccurately place a limb, the limb falls through one of the openings in the grid, which is counted as a foot fault. The total number of steps (movement of each forelimb) that the animal used to cross the grid and the total numbers of foot faults for left forelimb were recorded. The mNSS and foot-fault test were carried out before TBI and at 1, 7, 14, 21 and 25 days after TBI or surgery. After the behavioral tests, animals of each control/treatment groups were sacrificed, transcardially perfused at the 25-day time point, and brains were collected for immunohistochemistry (IHC) analysis. FIG. 2A shows that significantly improved sensorimotor scores were measured at Days 14-25 after TBI in the SEQ ID NO. 5-treated group compared to the saline-treated group (P<0.05). The Morris water maze protocol in the present study was used to detect spatial learning and memory deficits. The more time the rats spent in the correct quadrant, the better the spatial learning and memory. As shown in FIG. 2B, the percentage of time spent in the correct quadrant (Northeast) by all TBI rats gradually increased from Days 21 to 25 after surgery. As compared to the control (saline-treated) group, the SEQ ID NO. 5 treated group showed a significant improvement in spatial learning and memory retention at Days 23 to 25 post-injury (3rd to 5th day of MWM testing) (P<0.05). As compared to preinjury baseline, the incidence of contralateral right forelimb footfaults significantly increased after TBI. Treatment with SEQ ID NO. 5 showed no significant improvement in neurological function over the control group, assessed by the footfault test (see FIG. 2C).

The main findings, summarized in of the present in vivo efficacy study are: 1) posttraumatic SEQ ID NO. 5 treatment (starting at 24 h with one additional injection at 48 h) after TBI provides similar and significant long-term (at least 1 month) improvements in sensorimotor functional recovery and spatial learning, as evaluated by mNSS score and MWM tests, respectively; and 2) the long-term improvements in spatial learning and sensorimotor function may mainly derive from SEQ ID NO. 5-promoted neurovascular remodeling including increased angiogenesis, cell proliferation and neurogenesis. Many treatments in experimental TBI initiated immediately or within 6 hours after TBI show neuroprotection. However, the present short-term study (25-day survival) in rats demonstrates that delayed (24 h) treatment with SEQ ID NO. 5 not only significantly improves spatial memory and learning after controlled cortical impact (CCI) but also promotes sensorimotor functional recovery (reduced mNSS score) indicating that the therapeutic time window may not be limited to early hours but can be extended to later time periods after TBI. We also observed significant brain remodeling including increased cell proliferation, angiogenesis and neurogenesis promoted by SEQ ID NO. 5. The SEQ ID NO. 5-promoted brain remodeling may play an important role in functional recovery after TBI. Although the nervous and vascular systems are functionally different, they show a high degree of anatomic parallelism and cross-talk. The realization that both systems use common genetic pathways not only forms a link between vascular biology and neuroscience, but also promises to accelerate the discovery of new mechanistic insights and therapeutic opportunities. Under normal conditions, adult neurogenesis in the subgranular zone (SGZ) of the DG and subventricular zone (SVZ) takes place within an angiogenic microenvironment. In vivo, neurogenesis and angiogenesis are highly interdependent and work together to promote brain remodeling and subsequent improvement of neurological functional recovery after brain injury. In the present short-term (25-day) study, newborn mature neurons were found in the injury boundary zone, which probably came from neuroblasts migrating from the SVZ to the injury boundary zone after injury and treatment. Our present data indicate that newborn neurons in the DG may participate in brain repair and functional recovery. It is unknown whether additional doses of SEQ ID NO. 5 increase the survival rate of newborn neurons in the injured brain, and further studies are necessary. In conclusion, posttraumatic SEQ ID NO. 5 therapy for TBI significantly enhances angiogenesis and neurogenesis, and improves sensorimotor function and spatial learning recovery, suggesting that treatment with SEQ ID NO. 5 provides both neuroprotective and neurorestorative (that is, neurovascular remodeling) effects.

While the present invention has been particularly shown and described with reference to the foregoing preferred and alternative embodiments, it should be understood by those skilled in the art that various alternatives to the embodiments of the invention described herein may be employed in practicing the invention without departing from the spirit and scope of the invention as defined in the following claims. It is intended that the following claims define the scope of the invention and that the method and apparatus within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

1. A method of inhibiting neuronal damage in a patient following traumatic brain injury comprising:
administering at least one effective dose of a peptidomimetic calpain inhibitor to the upper one-third of the patient's nasal cavity, thereby enabling the at least one effective dose of peptidomimetic calpain inhibitor to bypass the patient's blood-brain barrier and delivering the at least one effective dose of peptidomimetic calpain inhibitor to the patient's central nervous system; and
treating the traumatic brain injury.

2. The method of claim 1 wherein the at least one effective dose of peptidomimetic calpain inhibitor is administered to the upper one-third of the patient's nasal cavity as at least one of a liquid spray, a powdered spray, nose drops, a gel, an ointment, or a combination thereof.

3. The method of claim 1 wherein the peptidomimetic calpain inhibitor is a drug comprising a synthetic peptide of SEQ ID NOS: 2, 3, 5, 6, 7, 8, 9, or 10 and a pharmaceutically acceptable excipient or other additive selected from the group consisting of a hydrophobic blood-brain-barrier or a membrane-permeant amino acid sequence linked by a polyethylene glycol linker to the N-terminus of said peptide, SEQ ID NO. 11, SEQ ID NO. 12, a nonaqueous vehicle, nanoparticle encapsulation, a solvent, a diluent, adjuvant, a chelating agent, a stability enhancing additive, antimicrobial preservatives, antioxidants, buffers, an antibacterial or an antifungal agent, parabens, chlorobutanol, phenol, sorbic acid, an isotonic agent, a sugar, sodium chloride, a prolonged absorption agent, aluminum monostearate, a gelatin, or a combination thereof.

4. The method of claim 3 wherein the synthetic peptide is that of SEQ ID NO: 5.

5. The method of claim 3 wherein the synthetic peptide is that of SEQ ID NO: 2 or SEQ ID NO: 5 wherein the peptidic bond(s) of one or more of the following amino acid pairs in the sequence is replaced with a non-peptidic reduced amide bond: Ser4-Ser5, Tyr6-Ile7, Lys13-Arg14, Glu15-Val16, Lys21-Tyr22, Tyr22-Arg23.

6. The method of claim 3 wherein the synthetic peptide is that of SEQ ID NO: 2, wherein there is side-chain cyclization between Lys21 and Glu24.

7. The method of claim 1 wherein the at least one effective dose of peptidomimetic calpain inhibitor is 0.0001 to 1.0 mg/kg.

8. (canceled)

9. (canceled)

10. The method of claim 1 further comprising administering the at least one effective dose of peptidomimetic calpain inhibitor until the concentration of peptidomimetic calpain inhibitor in the patient's brain is within the range of 0.1 nM to 50 μM.

11. The method of claim 1 wherein the administration of the at least one effective dose of peptidomimetic calpain inhibitor treats neurodegeneration or inhibits memory loss or treats physical impairment or treats behavioral impairment in the patient, wherein the neurodegeneration is caused by the traumatic brain injury.

12-14. (canceled)

15. The method of claim 1 wherein the traumatic brain injury is caused by a head injury.

16. A traumatic brain injury induced neuronal damage inhibiting neurotherapeutic composition comprising:
at least one effective dose of a peptidomimetic calpain inhibitor having a synthetic peptide of SEQ ID NOS: 2, 3, 5, 6, 7, 8, 9, or 10.

17. The composition of claim 16 further comprising a pharmaceutically acceptable excipient or other additive, said pharmaceutically acceptable excipient or other additive being at least one of a liquid spray, a powdered spray, nose drops, a gel, an ointment, or a combination thereof.

18. The composition of claim 17 wherein the pharmaceutically acceptable excipient or other additive is selected from the group consisting of a hydrophobic blood-brain-barrier or a membrane-permeant amino acid sequence linked by a polyethylene glycol linker to the N-terminus of said peptide, SEQ ID NO. 11, SEQ ID NO. 12, a nonaqueous vehicle, nanoparticle encapsulation, a solvent, a diluent, adjuvant, a chelating agent, a stability enhancing additive, antimicrobial preservatives, antioxidants, buffers, an antibacterial or an antifungal \agent, parabens, chlorobutanol, phenol, sorbic acid, an isotonic agent, a sugar, sodium chloride, a prolonged absorption agent, aluminum monostearate, a gelatin, or a combination thereof.

19. The composition of claim 16 wherein the synthetic peptide is that of SEQ ID NO: 5.

20. The composition of claim 16 wherein the synthetic peptide is that of SEQ ID NO: 2 or SEQ ID NO: 5 and wherein the peptidic bond(s) of one or more of the following amino acid pairs in the sequence is replaced with a non-peptidic reduced amide bond: Ser4-Ser5, Tyr6-Ile7, Lys13-Arg14, Glu15-Val16, Lys21-Tyr22, Tyr22-Arg23.

21. The composition of claim 16 wherein the synthetic peptide is that of SEQ ID NO: 2, wherein there is side-chain cyclization between Lys21 and Glu24.

22. The composition of 18 claim 16 wherein the at least one effective dose of peptidomimetic calpain inhibitor is 0.0001 to 1.0 mg/kg.

23. (canceled)

24. The composition of claim 16 further comprising the at least one effective dose of peptidomimetic calpain inhibitor having a volume of 0.015 to 1.0 ml.

25-28. (canceled)


SEQUENCE LISTING (12 sequences)

SEQ ID NO: 1 - DNA, 26 bp, Artificial sequence
  Synthetic
ctccaaaatatagggaactattggct

SEQ ID NO: 2 - protein, 27 aa, Artificial sequence
  Synthetic - calpastatin-derived peptide
DPMSSTYIEELGKREVTIPPKYRELLA

SEQ ID NO: 3 - protein, 27 aa, Artificial sequence
  Synthetic-calpastatin-derived peptide
DPMSSTYIEELGKREVTIPPKYVALLP

SEQ ID NO: 4 - protein, 9 aa, Artificial sequence
  Synthetic-calpastatin-derived peptide
AVLLALLAP

SEQ ID NO: 5 - protein, 36 aa, Artificial sequence
  Synthetic-calpastatin-derived peptide
DPMSSTYIEELGKREVTIPPKYVALLPAVLLALLAP

SEQ ID NO: 6 - protein, 27 aa, Artificial sequence
  Synthetic-calpastatin-derived peptide
  MUTAGEN (18)..(18): Nva
  MUTAGEN (23)..(23): Orn
DPMSSTYIEELGKREVTNPPKYNELLA

SEQ ID NO: 7 - protein, 27 aa, Artificial sequence
  Synthetic-calpastatin-derived peptide
  MUTAGEN (18)..(18): Nva
DPMSSTYIEELGKREVTNPPKYRELLA

SEQ ID NO: 8 - protein, 27 aa, Artificial sequence
  Synthetic-calpastatin-derived peptide
  MUTAGEN (23)..(23): Orn
DPMSSTYIEELGKREVTIPPKYNELLA

SEQ ID NO: 9 - protein, 27 aa, Artificial sequence
  Synthetic-calpastatin-derived peptide
  MUTAGEN (18)..(18): Nva
DPMSSTYIEELGKREVTNPPKYVALLP

SEQ ID NO: 10 - protein, 36 aa, Artificial sequence
  Synthetic-calpastatin-derived peptide
  MUTAGEN (18)..(18)
DPMSSTYIEELGKREVTNPPKYVALLPAVLLALLAP

SEQ ID NO: 11 - DNA, 48 bp, Artificial sequence
  Synthetic-blood brain barrier permeant enconding sequence
gccgcggtagcgctgctcccggcggtcctgctggccttgctggcgccc

SEQ ID NO: 12 - protein, 16 aa, Artificial sequence
  Synthetic
AAVALLPAVLLALLAP